(19) United States
(12) Patent Application Publication
(10) Pub. No.: US 2026/0344995 A1
(43) Pub. Date: Nov. 5, 2026

(54) SYSTEM FOR PROVIDING ON-DEMAND PRESCRIPTION EYEWEAR

(71) Applicants: Steven Maxfield, McMinniville, OR (US); Daniel Miller, McMinniville, OR (US)

(72) Inventors: Steven Maxfield, McMinniville, OR (US); Daniel Miller, McMinniville, OR (US)

(21) Appl. No.: 19/112,569

(22) PCT Filed: Apr. 4, 2024
(86) PCT No.: PCT/US2024/022948

§ 371 Date: Mar. 17, 2025

Related U.S. Application Data
(60) Provisional application No. 63/457,050, filed on Apr. 4, 2023.

(51) Int. Cl. A61B 3/00; A61B 3/14; G02C 7/02; G06V 10/75; G06V 10/82; G06V 40/16; G06V 40/18; G16H 40/63

(52) U.S. Cl. CPC A61B 3/0025; A61B 3/145; G02C 7/027; G06V 10/751; G06V 10/82; G06V 40/165; G06V 40/166; G06V 40/171; G06V 40/18; G16H 40/63; G06V 2201/03

(57) ABSTRACT

The invention provides a system and method for on-demand provision of prescription eyewear to a user. The system can include an interface with a camera, an eye measurement machine, a neural network implemented by computing processing units, and a database of standard lenses and eyeglass frames. The camera captures representative images of the user's facial parameters, which are analyzed by the neural network to identify facial structures and compare them with a facial feature database. The method involves capturing representative images, utilizing the eye measurement machine, implementing the neural network, and employing the lens and frame databases to provide the recommended eyewear. The system and method can also maintain a database of autorefraction data and subjective refraction data to optimize the autorefraction based on known parameters about the patient.

### CROSS REFERENCE TO RELATED APPLICATION

This application claims benefit from currently pending U.S. Provisional Application No. 63/457,050 titled “System for Providing On-Demand Prescription Eyewear” and having a filing date of Apr. 4, 2023, and all of which is incorporated by reference herein.

### FIELD OF THE INVENTION

The present invention relates to systems and methods for providing on-demand prescription eyewear to a user. More specifically, the invention pertains to a system that utilizes a neural network capturing facial parameters and eye measurements of the user, identifies facial structures, and recommends suitable lens and frame combinations based on the analyzed data.

### BACKGROUND OF THE INVENTION

Half of the United States population has visually significant refractive error (“VSRE”). Individuals with VSRE are considered impaired without glasses. Rates of myopia, a type of VSRE, are growing and access to viable care is not keeping pace. With this comes a growing number of people who are unable to access appropriate vision care and obtain spectacle correction. When an impairment is not corrected it is defined as an uncorrected refractive error (URE). In 2015, 8.2 million people in the U.S. had visual impairment due to URE and this is estimated to double by 2050. With access to glasses, these people would no longer experience visual impairment. Multiple studies have shown negative health, educational, and economic effects from visual impairment. Low-income and poor access in rural settings limits people's ability to obtain visual correction and thus progress in education and employment.

The current methods for providing prescription eyewear to users often involve a series of manual measurements and fittings. These methods can be time-consuming, inconvenient, and prone to human error. For instance, the measurement of facial parameters such as pupillary distance, bridge width, temple-to-temple distance, and temple length are typically done manually by an optician. These measurements are crucial for the proper fitting of eyeglasses, as incorrect measurements can lead to discomfort and poor vision correction. Moreover, the selection of suitable eyeglass frames and lenses is often a trial-and-error process, relying heavily on the user's subjective feedback. This process can be overwhelming for the user due to the vast array of options available. Furthermore, the final selection may not always be the most optimal in terms of fit and vision correction.

In addition, current systems for providing prescription eyewear do not leverage the advancements in technology such as machine learning and computer vision. Autorefractors, often found as tabletop devices, gauge a patient's refractive error and ascertain their eyeglass prescription. However, their operation mandates skilled technicians, with validation by eyecare professionals. These types of technologies have the potential to automate and improve the accuracy of the measurement and selection process. For instance, a neural network can be trained to identify facial structures from images or videos and compare them with a database of facial images. This can potentially lead to more accurate measurements and better fitting eyewear. Furthermore, current systems do not provide on-demand provision of prescription eyewear. This means that users have to wait for their eyewear to be prepared and delivered, which can take several days or even weeks. This delay can be inconvenient for users, especially those who need their eyewear urgently.

Therefore, there is a need for a system that can provide on-demand prescription eyewear to users. Such a system should be able to accurately measure the user's facial parameters and eye measurements, recommend suitable lenses and frames, and provide the eyewear in a timely manner. The system should also leverage advancements in technology such as machine learning and computer vision to improve the accuracy and efficiency of the process.

### BRIEF SUMMARY OF THE INVENTION

The present invention provides among other things an on-demand prescription eyewear to a user. The system can comprise an interface with a headrest and at least one camera that captures at least one representative image of at least one facial parameter of the user. An eye measurement machine coupled to a platform that allows movement in the x, y, and z direction relative to the user's eyes, uses the representative image to scan the user's facial parameters and position itself for taking at least one eye measurement and at least one frame measurement.

The system can include a neural network implemented by one or more computing processing units. The neural network identifies at least one facial structure from the representative image, then searches for other facial features including eyes and pupils and identifies their location in the image which can be done by using layers in the neural network that have nodes that have been trained to identify faces and facial features in images using labeled images for training. The system further includes a lens database of standard lenses and a frame database of standard eyeglass frames. These databases provide at least one recommended lens and frame combination to the user based on the analyzed facial parameters and eye measurement.

The method for on-demand provision of prescription eyewear involves capturing at least one representative image of at least one facial parameter of the user through an interface equipped with a headrest and at least one camera. An eye measurement machine, capable of moving in the x, y, and z directions relative to the user's eyes, uses the representative image to scan the user's facial parameters and position itself for obtaining at least one eye measurement.

The method can further include implementing a neural network to identify at least one facial structure from the representative image and compare it with at least one database image and then can identify additional facial features such as eyes and pupils. The neural networks can use trained nodes and layers to identify these features in images. The nodes and layers can be trained on test images, which include images that have facial features labeled.

The method can also involve employing a first database of standard lenses and a second database of standard eyeglass frames to provide at least one recommended lens and frame combination to the user based on the analyzed facial parameters and the at least one eye measurement. The method can also include maintaining a database of autorefraction data and subjective refraction data to optimize the auto refraction based on known parameters about the patient. The autorefraction measurement may be provided to a healthcare professional who can remotely issue a prescription based on the at least one eye measurement.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors'intent and desire that the simple, plain and ordinary meaning to the terms be applied to the interpretation of the specification and claims. Aspects and applications of the invention presented here are described below in the drawings and detailed description of the invention.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of . . . ”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF THE DRAWINGS

A more complete understanding of the present invention may be derived by referring to the detailed description when considered in connection with the following illustrative figures. In the figures, like reference numbers refer to like elements or acts throughout the figures.

FIG. 1 shows the flow process of the system for providing on-demand prescription eyewear;

FIG. 2 shows the facial parameters taken for the system for providing on-demand prescription eyewear; and

FIG. 3 shows an isometric view of the system for providing on-demand prescription eyewear.

Elements and acts in the figures are illustrated for simplicity and have not necessarily been rendered according to any particular sequence or embodiment.

### DETAILED DESCRIPTION

In the following description, and for the purposes of explanation, numerous specific details are set forth to provide a thorough understanding of the various aspects of the invention. It will be understood, however, by those skilled in the relevant arts, that the present invention may be practiced without these specific details. In other instances, known structures and devices are shown or discussed more generally to avoid obscuring the invention. In many cases, a description of the operation is sufficient to enable one to implement the various forms of the invention, particularly when the operation is to be implemented in software. It should be noted that there are many different and alternative configurations, devices, and technologies to which the disclosed inventions may be applied. The full scope of the inventions is not limited to the examples that are described below.

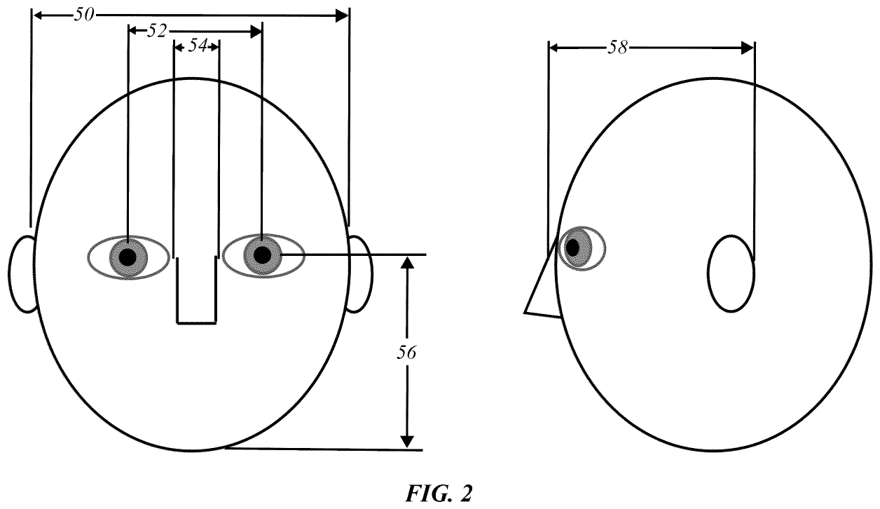

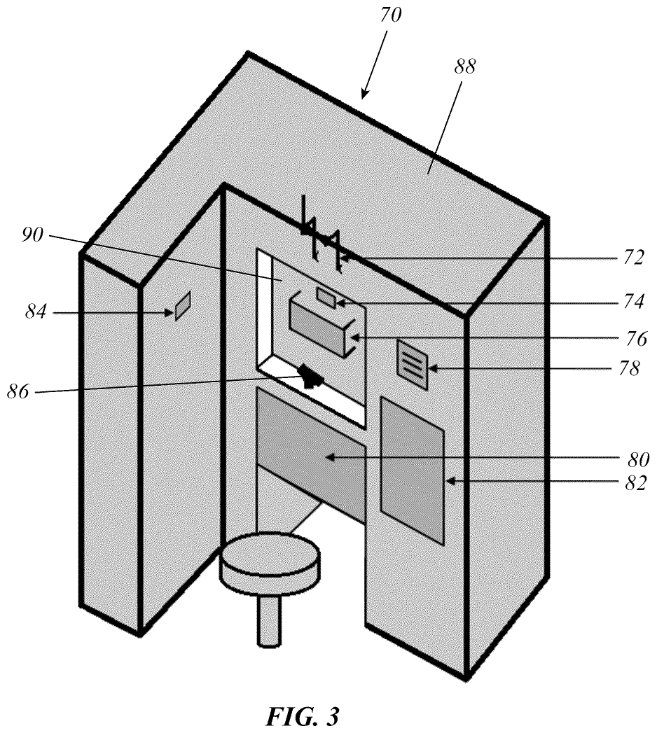

The present invention relates to a system and method for providing on-demand prescription eyewear to a user. Referring to FIG. 3, the system is shown generally at 70 wherein the system for providing on-demand prescription eyewear to a user can comprise an interface 88 equipped with a headrest 86 and chin rest 86 and at least one camera 74, 84. The camera 74, 84 can capture at least one representative image of at least one facial parameter of the user. The facial parameters as shown in FIG. 2 can be such as, for example, but are not limited to, facial width 50, pupillary distance 52, bridge width 54, pupil to chin 56, facial width 58, temple length 58 (the distance from the bridge of the nose to the ear), or the like. These parameters are crucial in determining the correct fit and comfort of the eyewear for the user. The camera 74 can be such as, for example, RGB camera, infrared camera, depth camera, facial recognition camera, machine vision camera, 360-degree camera, laser scanning camera, AI-powered camera, or the like. The camera 74 can be a video camera or a still camera capable of taking a complete 360-degree scan of the user's facial parameters. The system can have more than one camera positioned around the user or can be moved in the x, y, z-axis by at least one motor which can move the camera around the user capturing at least one of the user's facial parameters.

In the case the camera 74, 84 is a video camera, the representative image can be a continuous scan or a plurality of images of the user's facial parameters. The continuous scan allows for a more comprehensive and detailed capture of the user's facial parameters. In certain embodiments, the system can be a combination of video and plurality of images wherein the plurality of images can provide the advantage of capturing different angles and aspects of the user's face, which can be beneficial in accurately determining the facial parameters. The camera 84 can be positioned anywhere on the interface 88 but in the preferred embodiment the camera is at the user's height while sitting can be adjusted for varying user's heights.

The system 70 can further comprise an eye measurement machine 76 that can be coupled to a platform 90. This platform 90 allows the eye measurement machine 76 to move in the x, y, and z direction relative to the user's eyes. The platform 90 can be coupled to at least one motor which can move the eye measurement machine 76 in any combination of the x, y, and z axis. The eye measurement machine 76 can be such as, for example, autorefractors, phoropters, keratometer, tonometer, optical biometers, retinal cameras, wavefront aberrometer, optical coherence tomography machine, or the like. The eye measurement machine 76 can measure the refractive state of the eye and can measure the eyes dimension such as the axial length, anterior chamber depth, lens thickness and can determine if the user has at least one of such as, for example, corneal astigmatism, retinal disease, glaucoma, or the like. The camera 74 can scan the user's facial parameters and can position the eye measurement machine for taking an eye measurement of at least one eye and can then position itself to take at least one measurement of the user's other eye.

The system 70 can further comprise a display 82, a speaker 78 and headrest 72 which can be coupled to the interface 88. The display 82 can be such as, for example, touchscreen display, liquid crystal display, plasma display, light-emitting diode display or the like. The display 82 can interact with the user and allow the user or the doctor to see the lens, frame and measurement information taken from the user. The speaker 78 can be coupled to the interface and can allow the user to hear commands to adjust their face, head, eye position or the like. The speaker 78 can be such as, for example, compact speaker, Bluetooth speaker, studio speaker, horn speaker, or the like. The headrest 72 can be coupled to interface above the camera 74 and can be adjustable to fit the user's head size. The headrest 72 can be adjusted vertically, horizontally and the depth from the interface 88 wherein the headrest can be manually adjusted or mechanically adjusted in the headrest's x, y, and z location. The headrest 72 can automatically or manually fold down in front of the user and can be contoured to fit the user's head and can have padding.

The system can further comprise a neural network implemented by one or more computing processing units. The neural network can identify at least one facial parameter or structure from the representative image using layers, nodes, and its tuning parameters from being trained off labeled facial features in images. The neural network can be a convolutional neural network, which is commonly used for analyzing and interpreting images. The convolutional neural network can extract facial features or the facial structure of the user from at least one image that is fed as input, it then can use the convolutional layers to pass information about the image and extract different features such as edges, shapes. It then can analyze the feature and find and extract that information from the image to output feature location and metadata. The neural network can be trained by using labeled images that highlight wanted features for extraction. The neural network then can create layers and nodes to analyze new images for wanted features. In certain embodiments, the convolutional neural network which creates a facial layer from the facial parameters of the user with a facial database layer from the at least one facial database image analyzing the layers and finding at least one matching feature between the facial layer and the facial database layer wherein a frame database layer is analyzed and compared to the standard frame sizes matching the user's facial parameters to a matching frame.

The neural network can be composed of a forward-fed input layer which receives an image for processing. The neural network can then have a filter to perform preprocessing on an image, making it easier for analysis. The network can then have additional layers of hierarchical or pooling where features of the faces are detect distinctly then pooled to identify a face, within that face other features such as pupils to create more efficient processing. In addition, to find the user's pupil center and for horizontal alignment, the user's eye can be scanned and an image is taken of the user's eye, the image can be converted into black and white wherein the neural network can identify black circles which can be the pupil and then locate the center point of the black circle or user's pupil. In other embodiments the pupil center can be replaced by targeting the center of the cornea. The pupil center, visual axis and corneal center are usually slightly off from each other but are all within a fraction of a millimeter from each other. For vertical alignment, a midpoint between the upper and lower lid can be used.

The data taken from the eye measurement machine can be transferred from the eye measurement machine to the one or more computing processing units having at least one memory module wherein the neural network can assign the eye measurements at least one eye layer for each eye wherein the eye layer can be one of the input layers within the neural network. The eye layer can comprise of such as, for example, refractive error of the eye, spherical refraction, cylindrical refraction, axis, pupil distance, cycloplegic refraction, keratometry, pupillary distance and segmented height, wavefront aberrations, corneal topography, aberrometry or the like. The identified eye features can be compared to an eye database wherein the eye database can have standard eye measurements that can be constantly updated and referenced.

The neural network can further comprise a feedback process that passes the image layer through the filter, convolutional, and pooling layers wherein each layer can transform the data until the final output is produced. The convolutional neural network has the capability to generate a convolutional layer based on the user's facial parameters, producing facial features and their corresponding locations. These features are then cross-referenced with a frame database to analyze and compare them to standard frame sizes, thereby identifying a matching frame suited to the user's facial dimensions. In alternative embodiments, a facial database layer derived from at least one facial database image undergoes analysis to detect matching features between the facial layer and the facial database layer. Simultaneously, a frame database layer is scrutinized and juxtaposed against standard frame sizes to find a suitable match for the user's facial parameters. The frame can be such as, for example, full rim frame, half rim frame, rimless frame, round frame, oval frame, browline frame, contact lenses, or the like. The display 82 can allow the user to choose from a list of frames that would be fit the results from the measurements taken. In certain embodiment, the convolutional neural network can further comprise at least one eye measurement layer wherein the eye measurement layer can analyzed and compared to at least one lens database layer wherein the interface notifies the user that there is a matching lens, frame, or to contact a doctor.

In embodiments, the system can also comprise a lens database of standard lenses and a frame database of standard eyeglass frames wherein the lens database can be assigned a lens layer, and the frame database can be assigned a frame layer within the neural network. Based on the facial parameters and eye measurements, the system can provide at least one recommended lens and frame combination to the user. The lens and frame recommendation can be made by analyzing and comparing the user's facial parameters and eye measurements with the standard lens sizes and eye specifications in the lens, frame and eye databases.

The method for on-demand provision of prescription eyewear involves capturing at least one representative image of at least one facial parameter of the user through the interface equipped with a hard rest and at least one camera The eye measurement machine, which are capable of moving in the x, y, and z directions relative to the user's eyes, is then positioned based on the representative image. An autorefraction measurement of the patient is taken when the eye measurement machine is in the desired location relative to the user's eyes.

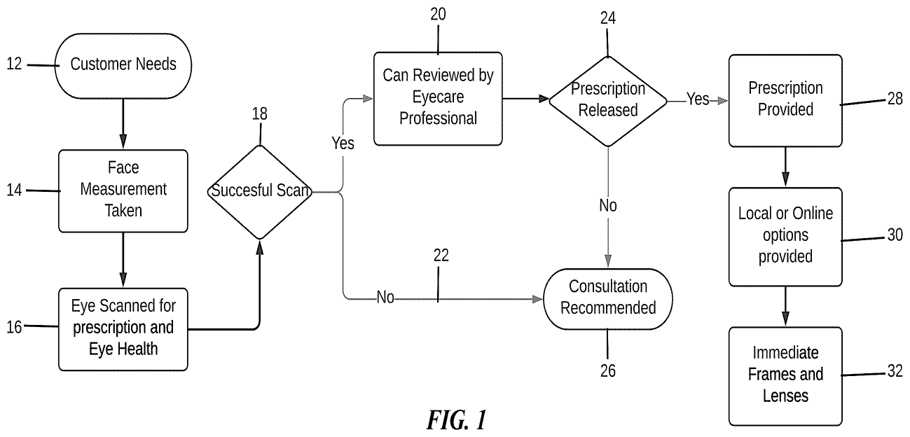

Referring to FIGS. 1 and 3, at 12 the customer is in need of eyeglasses, the customer sits down at the system and places their forehead into the headrest 72. The customer's face measurements are taken by the at least one camera 74, 84 at 14. The eye measurement machine is then moved into position to scan the user's eye. The customer's eye is scanned for prescription and for eye health at 16. If the scan was successful 18, then the scan can be automatically approved within certain parameters or reviewed by an eyecare professional either remotely or at the system's physical location 20, and the physician can release the prescription 24. The system gets the approval and scans the database for an acceptable lens and frame that will fit the customer. If the scan is not successful 22 then the system recommends the customer to get a consultation from a physician 26 or if the prescription is not released from the physician, then a consultation is recommended 26. If the prescription is released 24 then the prescription is provided 28 and a local or online frames and lens are scanned for options and those options are giving to the user from the database 30. If the lens and frame are available, then the customer is matched with and given frames with lenses 32.

The method further involves implementing a neural network that identifies at least one facial structure from the representative image and uses its tuned nodes and layers to output the location of wanted features. A first database of standard lenses and a second database of standard eyeglass frames are employed to provide at least one recommended lens and frame combination to the user based on the analyzed facial parameters and the at least one eye measurement.

The method also involves maintaining a database of autorefraction data and subjective refraction data. This database is used to optimize the autorefraction based on known parameters about the patient. These parameters can include the patient's age, biotype, and level of astigmatism. The database can also include autorefraction data and subjective refraction data taken over time for a single patient wherein the subjective refraction data can include at least one of the patient's age, ethnicity, prior ocular surgery, historical refractive date, and level of astigmatism. This longitudinal data can provide valuable insights into the changes in the patient's eye parameters over time and can help in making more accurate and personalized eyewear recommendations. The autorefraction measurement is provided to a healthcare professional wherein if the user's measurements fall within acceptable reliability parameters, the healthcare professional may choose to allow automatic prescription without review. The method involves providing a reliability score for the at least one eye measurement that relates to how reliable the at least one eye measurement is to the patient's hypothetical subjective refraction data determined from information obtained by the system. The method further involves adjusting the autorefraction measurement based on the database data to improve the accuracy of the autorefraction measurement.

The method also utilizes reliability metrics based on the information obtained by the eye machine and this can be used to determine if a prescription generated from the eye machine is likely to be accurate. This reliability score, for example, can be generated from analyzing the regularity of mire rings in an autorefraction system or the degree of higher order aberrations from a wavefront aberrometer. The autorefraction measurement can be adjusted based on the database data to improve the accuracy of the autorefraction measurement. The at least one facial parameter can include at least one of the bridge width to temple distance, facial width, pupillary distance, and temple length. The at least one representative image can be such as, for example, video, photograph, scan, plurality of photos, or the like.

In conclusion, the present invention provides a system and method for providing on-demand prescription eyewear to a user. The system and method involve capturing at least one representative image of at least one facial parameter of the user, utilizing an eye measurement machine to obtain at least one eye measurement, implementing a neural network to identify at least one facial structure from the representative image, and employing a lens and frame database to provide at least one recommended lens and frame combination to the user based on the analyzed facial parameters and the at least one eye measurement. The system and method provide a convenient and efficient way for users to obtain prescription eyewear.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

1. A system for providing on-demand prescription eyewear to a user comprising:
an interface having at least one camera wherein the at least one camera takes at least one representative image of at least one facial parameter of the user;
an eye measurement machine coupled to a platform that allows the eye measurement machine to move in the x, y and z direction relative to the user's eyes wherein the representative image scans the user's facial parameters moving the eye measurement machine in position for taking at least one eye measurement;
a neural network implemented by one or more computing processing units wherein the neural network identifies at least one facial structure from the at least one representative image and compares the at least one facial feature image with at least one facial feature database image; and
a lens database of standard lenses and a frame database of standard eye glass frames providing at least one recommended lens and frame combination to the user based on the facial parameters and eye measurement.

2. The system of claim 1, wherein the facial parameter is at least one of a pupillary distance, a bridge width, a facial width, and a temple length of the user.

3. The system of claim 1, wherein the one or more computing processing units creates a 3D rendering of the user facial structure comprising of facial X, Y, and Z coordinates and sending the information to the eye measurement machine for initial centration over the right pupil.

4. The system of claim 1, wherein the eye measurement machine is an autorefractor or wavefront aberrometer.

5. The system of claim 1, wherein the at least one camera is a video camera or a still camera.

6. The system of claim 1, wherein the at least one representative video is a continuous scan or a plurality of images.

7. The system of claim 2, wherein the neural network comprises a convolutional neural network which creates a facial layer from the facial parameters of the user with a facial database layer from the at least one facial database image analyzing the layers and finding at least one matching feature between the facial layer and the facial database layer wherein a frame database layer is analyzed and compared to the standard frame sizes matching the user's facial parameters to a matching frame.

8. The system of claim 7, wherein the convolutional neural network further comprises at least one eye measurement layer wherein the eye measurement layer is analyzed and compared to at least one lens database layer wherein the interface notifies the user that there is a matching lens or to contact a doctor.

9. A method for on-demand provision of prescription eyewear to a user, comprising:
capturing at least one representative image of at least one facial parameter of the user through an interface equipped with a headrest and at least one camera;
utilizing an eye measurement machine coupled to a platform capable of moving in the x, y, and z directions relative to the user's eyes, wherein the representative image scans the user's facial parameters and positions the eye measurement machine for obtaining at least one eye measurement based on the representative image; and
taking an autorefraction measurement of the patient when the eye measurement machine is in a desired location relative to the user's eyes.

10. The method of claim 9 further comprising implementing a neural network through one or more computing processing units, wherein the neural network identifies at least one facial structure from the representative image and uses the at least one facial structure to identify facial features such as eyes and pupils.

11. The method of claim 9, further comprising employing a first database of standard lenses and a second database of standard eyeglass frames to provide at least one recommended lens and frame combination to the user based on the analyzed facial parameters and the at least one eye measurement.

12. The method of claim 9, further comprising maintaining a database of autorefraction data and subjective refraction data to optimize the autorefraction based on known parameters about the patient.

13. The method of claim 12, wherein at least one of the autorefraction data and the subjective refraction data includes at least one of the patient's age, ethnicity, prior ocular surgery, historical refractive date, and level of astigmatism.

14. The method of claim 12 wherein the database includes autorefraction data and subjective refraction data taken over time for a single patient.

15. The method of claim 9 further comprising providing the autorefraction measurement to a healthcare professional and having the healthcare professional remotely issue a prescription based on the at least one eye measurement.

16. The method of claim 12 further comprising providing a reliability score for the at least one eye measurement that relates to how reliable the at least one eye measurement is to the patient's hypothetical subjective refraction data determined from information obtained by a system comprising:
an interface having at least one camera wherein the at least one camera takes at least one representative image of at least one facial parameter of the user;
an eye measurement machine coupled to a platform that allows the eye measurement machine to move in the x, y and z direction relative to the user's eyes wherein the representative image scans the user's facial parameters moving the eye measurement machine in position for taking at least one eye measurement;
a neural network implemented by one or more computing processing units wherein the neural network identifies at least one facial structure from the at least one representative image and compares the at least one facial feature image with at least one facial feature database image; and
a lens database of standard lenses and a frame database of standard eye glass frames providing at least one recommended lens and frame combination to the user based on the facial parameters and eye measurement.

17. The method of claim 12 further comprising adjusting the autorefraction measurement based on the database data to improve the accuracy of the autorefraction measurement.

18. The method of claim 9 wherein the at least one facial parameter includes at least one of the bridge width to temple distance, facial width, pupillary distance, and temple length.

19. The method of claim 9 wherein the at least one representative image is video.
